(19) United States
(12) Patent Application Publication
(10) Pub. No.: US 2004/0193154 A1
(43) Pub. Date: Sep. 30, 2004

(54) Bone and cartilage implant delivery device

(75) Inventors: Neil C. Leatherbury, San Antonio, TX (US); Mark Q. Niederauer, San Antonio, TX (US); Fred B. Dinger III, San Antonio, TX (US); Joe D. Blandford, San Antonio, TX (US)

Correspondence Address: GREENLEE WINNER AND SULLIVAN P C, 5370 MANHATTAN CIRCLE, SUITE 201, BOULDER, CO, 80303, US

(73) Assignee: OsteoBiologics, Inc., San Antonio, TX (US)

(21) Appl. No.: 10/785,388

(22) Filed: Feb. 23, 2004

Related U.S. Application Data
(60) Provisional application No. 60/448,965, filed on Feb. 21, 2003.

(51) Int. Cl. A61B 17/56

(52) U.S. Cl. 606/53

(57) ABSTRACT

A method and device for inserting an implant of synthetic material or healthy bone or cartilage into a bone or cartilage defect of unknown depth. The device includes an inner shaft within a hollow outer shaft. One end of the inner shaft of the device is suitable for inserting into the bone or cartilage defect in order to determine the depth, while the other end of the outer shaft is suitable for holding an implant. The implant is cut to fit the defect once its depth has been determined and then inserted into the defect. A cutting device for cutting the implant is disclosed. Also disclosed is an implant capsule loader for inserting an implant into the delivery device.

### CROSS-REFERENCE TO RELATED APPLICATIONS

This application claims priority to U.S. Provisional Application No. 60/448,965 filed Feb. 21, 2003, which is incorporated herein in its entirety to the extent not inconsistent herewith.

### BACKGROUND OF THE INVENTION

This invention relates to an apparatus and methods for performing repairs of cartilage and bone defects.

It is well known in the art that implants can be inserted into damaged bone or cartilage layers to treat injuries to those tissue layers. One type of implant procedure involves inserting plugs of healthy bone or cartilage that are harvested from a healthy area of the patient's body and transplanted into the defect, as disclosed in U.S. Pat. No. 5,152,763 (Johnson et al.), U.S. Pat. No. 5,919,196 (Bobic et al.), and U.S. Pat. No. 6,358,253 (Torrie et al.). In the alternative an implant can consist of synthetic material, such as porous biocompatible foams or polymers, for example as disclosed in U.S. Pat. No. 4,186,448 (Brekke et al.), U.S. Pat. No. 5,607,474 (Athanasiou et al.), and U.S. Pat. No. 5,716,413 (Walter et al).

In implant procedures, defects of variable depths are often presented. In order for the implant, once inserted into the defect, to evenly match the surface of the surrounding tissue without protruding or forming a cavity, the depth of the defect must be determined and the length of the implant tailored to fit the defect. Generally, it is difficult to determine the exact depth of a defect and, therefore, to insert an implant with the correct length.

Current devices for inserting implants, either bone or cartilage transplants or synthetic materials, are deficient in determining defect depth. U.S. Pat. No. 5,782,835 (Hart et al.) teaches a bone plug emplacement tool comprising a cylinder with an internal bore along the longitudinal axis and a stem disposed for co-axial movement within the internal bore. A bone plug placed in the internal bore is delivered into the defect when the stem is advanced through the bore. However, the tool does not provide means for determining the depth of the defect or for tailoring the length of the implant to fit the defect.

U.S. Pat. No. 6,395,011 (Johanson et al.) similarly teaches a device comprising a push rod within a hollow cylinder for harvesting and implanting bone plugs. In addition, the device includes a translucent or transparent tip permitting the surgeon to view the bone plug during implantation. Although this is an improvement in that it allows the length of the bone plug to be determined after harvesting, it also does not provide means to determine the depth of the defect.

Absent an implant delivery device with means for determining defect depth, current methods of filling bone and/or cartilage defects include using a granular implant material to pack the defect, or using a separate plastic or metal depth gauge to measure the depth of the defect and then cutting the implant prior to insertion.

### SUMMARY OF THE INVENTION

The present invention provides a bone and/or cartilage implant delivery tool, which allows for measuring, sizing, and delivering of an implant to a bone and/or cartilage defect of unknown depth. Defects are not limited to bone and cartilage injuries. Defects can be intentionally created, such as the hole remaining in bone or cartilage tissue after a plug of healthy bone or cartilage is removed for transplantation. Intentionally created defects also include holes in bone or cartilage tissue created in order to insert autologous or allogenic grafts during ligament or tendon repair surgeries. This device is useful for arthroscopic repair of an osteochondral defect in a joint, such as a knee, and is also suitable for treatment of any bone or cartilage defect that is accessible by the device. Furthermore, the device is suitable for use with bone and cartilage transplants as well as synthetic implants. As used herein, “implant” includes implants made from synthetic materials and implants that are bone and cartilage transplants.

The delivery device of the present invention includes a tubular outer shaft having a proximal and a distal end and an internal bore along the longitudinal axis. In the present context, “proximal” refers to the end of the device initially oriented closest to the patient's body and used in measuring the depth of the defect as described below. “Distal” refers to the end of the device initially oriented away from the patient's body and used to contain the implant. The internal bore of the outer shaft is sized to accommodate the diameter of the implant or the profile of the implant if the implant is non-cylindrical.

A cylindrical inner shaft, also having proximal and distal ends, is disposed within the internal bore in the outer shaft, wherein the proximal end of the inner shaft is suitable for insertion into a defect. By “suitable for insertion into a defect” it meant that the proximal end of the inner shaft has a size and shape allowing it to fit within a bone and/or cartilage defect without distorting the defect or damaging the tissue layers. In one embodiment of the present invention, the proximal end of the inner shaft has a size and shape similar to the size and shape of the implant. The inner shaft has a diameter that also allows it to be slidably engaged with the outer shaft. “Slidably engaged” means the inner shaft can slide within the bore in the outer shaft. The inner shaft may be solid or have a cannula through its center.

The delivery device comprises means to provide friction-retarded movement of the inner shaft through the outer shaft. The inner shaft may have a “friction member”, which is herein defined as a section of the inner shaft having a diameter large enough to contact the inner surface of the outer shaft and provide a tight fit within the internal bore. The friction member may be coated with rubber or other materials to provide additional friction. The surfaces of the outer shaft and inner shaft also may be modified to provide friction-retarded movement. For example, a section of the outer shaft's inner surface may contain small beads and a corresponding section of the inner shaft's outer surface may contain small ridges. When the inner shaft is moved through the outer shaft, the small beads on the outer shaft contact the ridges on the inner shaft and provide additional friction. Alternatively, a section on the inner surface of the outer shaft may contain ridges or serrated teeth that engage ridges or serrated teeth disposed on the corresponding section on the outer surface of the inner shaft. When the inner shaft is moved through the outer shaft, the ridges and/or serrated teeth contact each other and movement is restricted. Other means that prevent unwanted movement of the inner shaft through the outer shaft include otherwise texturing the surfaces of the inner shaft and outer shaft, or coating the surfaces of the inner shaft and outer shaft with a viscous liquid.

When the inner shaft is disposed in the outer shaft so that the inner shaft does not protrude from the proximal end of the outer shaft, inserting an implant into the distal end of the outer shaft displaces the inner shaft towards the proximal end causing a portion of the inner shaft to protrude from the proximal end of the outer shaft. Conversely, when an implant is preloaded into the distal end of the outer shaft, the inner shaft is inserted in the proximal end of the outer shaft and advanced toward the distal end of the outer shaft until the distal end of the inner shaft contacts the implant. At this point, the implant will not extend beyond the distal end of the outer shaft and a portion of the inner shaft will protrude from the proximal end of the outer shaft.

With an implant at least partially inserted into the distal end of the outer shaft, the proximal end of the inner shaft is inserted into a defect of unknown depth. When the proximal end of the inner shaft contacts the bottom of the defect, the outer shaft is advanced towards the defect until the proximal end of the outer shaft contacts the surface of the tissue surrounding the defect. In relation to the outer shaft, this motion distally advances the inner shaft. As a result, the length of the inner shaft that protrudes from the proximal end of the outer shaft equals the depth of the defect. In addition, this motion displaces the implant in the outer shaft and causes a portion of the implant to extend beyond the distal end of the outer shaft.

The protruding end of the implant, i.e., the portion of the implant protruding from the distal end of the outer shaft, can be cut off with a knife or other cutting device. The remaining length of the implant in the distal end of the outer shaft equals the length of the inner shaft that protrudes from the proximal end of the outer shaft, which also equals the depth of the defect. The proximal end of the device is removed from the defect and the distal end of the device containing the implant is placed over the defect. The proximal end of the inner shaft, which is now the end furthest from the patient's body, is advanced towards the distal end of the outer shaft, which is now the end closest to the patient's body, pushing the implant into the defect.

A further embodiment of this invention includes the proximal and distal ends of the device having smooth, rounded edges to prevent damaging surrounding tissues. While the device can be constructed of any materials, including, but not limited to, medical grade plastic or metal, it is preferred that plastic is used to prevent scratching the bone or cartilage surface. In a further embodiment, a series of thin concentric slots cut into the outer surface of the outer shaft provide a gripping surface for easier handling of the device.

A further embodiment of this invention includes at least one slot or window in the distal end of the outer shaft of the device for visualizing the implant. The slot or window may be of any shape that allows the implant to be seen while the implant is disposed within the delivery device. The slot or window can also be covered with transparent material.

A further embodiment of this invention includes tapered leaves in the distal end of the outer shaft. Longitudinal slots are cut in the distal end of the outer shaft, creating opposing leaves. The leaves are the sections of the outer shaft between the longitudinal slots. These leaves can be made to taper slightly inward, creating slight compression on the implant to prevent undesired movement of the implant within the device.

A further embodiment of this invention includes a snap-bead feature on the distal end of the outer shaft for attaching items to the device. The snap-bead feature comprises an annular groove around the distal end of the outer shaft. An attachable item has one or more small beads or a rim that fits into this groove. One such attachable item is a temporary cap that fits over the distal end of the outer shaft to prevent accidental removal of the implant from the device.

In a further embodiment of this invention, the implant is delivered to a defect with bioactive fluids, such as blood, blood concentrate or cell suspension. After the implant has been sized and cut to fit the defect, a cap will be placed around the distal end of the outer shaft and bioactive fluids added via a window or slot. Additionally, a centrifuge can be used to load fluids and the delivery device can be made suitable for use in a centrifuge, i.e., structurally able to withstand the forces during centrifugation without leaking or damaging the implant, when loading fluids to the implant.

This invention also includes a cutting device comprising a cutting base having a hole adapted for receiving an implant protruding from the outer shaft of the implant delivery device and may also comprise at least one cutting blade for cutting off the portion of the implant that protrudes from the distal end of the outer shaft. “Adapted for receiving an implant” or “adapted for receiving the protruding end of an implant” with respect to the hole in the cutting base means the hole is big enough to allow the protruding end of the implant to pass through the hole, but at some point is small enough to prevent the distal end of the outer shaft from passing further through the hole. The point at which the hole allows the protruding end of the implant, but not the distal end of the outer shaft, to pass through is where the implant is cut. This point may be along the top or bottom surface of the cutting device base or somewhere within the cutting device base.

One embodiment of the implant cutting device comprises: a base comprising a vertical hole therethrough for receiving the protruding end of an implant and means for receiving at least one cutting blade; and at least one cutting blade adapted to slide within said means for receiving at least one cutting blade and cut off the protruding end of the implant. The “means for receiving a cutting blade” include a horizontal slot through the cutting device base or guides along the top or bottom surface of the base that allow the cutting blade to intersect the hole at the point where the implant but not the outer shaft can advance through the hole. The device may include a plurality (two or more) of cutting blades.

This invention also includes an implant capsule loader for inserting an implant into the shaft of an implant delivery device for delivery and orientation of multiple implants. The capsule loader comprises a hollow tube having a front end and a back end, adapted to fit within the distal end of the outer shaft of an implant delivery device. The capsule loader may also comprise a backplate disposed within said hollow tube covering the opening at the back end of said tube; and at least one flexible leaflet along the outer surface of said hollow tube fixed at the front end of said hollow tube and having a free end toward the back end of said hollow tube, said flexible leaflet having an outwardly extending prong at the free end thereof; said prong being adapted to fit within a hole in said shaft.

The terms “tube”, “tubular” and “cylindrical” used to describe the implant delivery device and implant capsule loader do not exclude depressions, reliefs, flats or flutes, or limit the shapes to only round cylinders. A tube is a hollow conduit, the cross-sectional area of which need not be circular or uniform along the length of the tube. The cross-sectional area of a tube can be any shape including, but not limited to, elliptical, hexagonal, octagonal, or irregular.

This invention also includes a kit comprising at least one implant delivery device. The kit may also include an implant and a knife or cutting device. The kit may comprise several implant delivery devices having different sizes of internal bores and inner shafts in order to accommodate defects and implants of varying sizes. The delivery devices of this kit can be individually color coded according to size.

### BRIEF DESCRIPTION OF THE DRAWINGS

FIG. 1 shows an implant delivery device of this invention with the inner shaft protruding from the proximal end of an outer shaft.

FIG. 2 shows the inner shaft of an implant delivery device of this invention.

FIG.3 shows the outer shaft of an implant delivery device of this invention.

FIG.4 shows a cross-sectional view of the implant delivery device of FIG. 1.

FIG. 5 shows an implant delivery device of this invention having longitudinal slots and a snap-bead feature on the distal end of the outer shaft with an inner shaft protruding from the proximal end of an outer shaft.

FIG. 6 shows the implant delivery device of FIG. 5 with an uncut implant disposed in the distal end of the outer shaft.

FIG. 7A is a cross-sectional side view of a cutting device of this invention with the distal end of the implant delivery device placed in the vertical hole therein. FIG. 7B is an exploded assembly view of the cutting device, also showing the distal end of the implant delivery device.

FIG. 8A is an end view of the inner shaft of the implant delivery device of FIG. 5 comprising a cannula. FIG. 8B is a side view of an inner shaft having ridges. FIG. 8C is an expanded view of the circled section of FIG. 8B showing the ridges in greater detail. The cannula in FIGS. 8B and 8C is shown by dotted lines.

FIG. 9A is an end view of the outer shaft of the implant delivery device of FIG. 5. FIG. 9B is a cross-sectional side view of the outer shaft shown in FIG. 9A. FIG. 9C is an expanded view of the circled section of FIG. 9B showing friction beads on the inner surface of the outer shaft.

FIG. 10A is an end view of a modified inner shaft of the implant delivery device of FIG. 5 comprising two alignment ribs. FIG. 10B is a side view of a modified inner shaft. FIG. 10C is an expanded view of the circled section of FIG. 10B showing serrated teeth along the surface of the inner shaft. The cannula in FIGS. 10B and 10C is shown by dotted lines.

FIG. 11A is an end view of a modified outer shaft of the implant delivery device of FIG. 5 comprising alignment slots. FIG. 11B is a cross-sectional side view of a modified outer shaft. FIG. 11C is an expanded view of the circled section of FIG. 11B showing serrated teeth on the inner surface of the outer shaft.

FIG. 12A shows cross-sectional view of an implant capsule loader containing an implant. The capsule loader is disposed within the outer shaft of the implant delivery device of FIG. 5. FIG. 12B shows an external view of an implant capsule loader of this invention. FIG. 12C shows a cross-sectional view of a capsule loader with the outer shaft of the implant delivery device after the inner shaft has pushed the implant out of the capsule loader and delivery device.

### DETAILED DESCRIPTION OF THE INVENTION

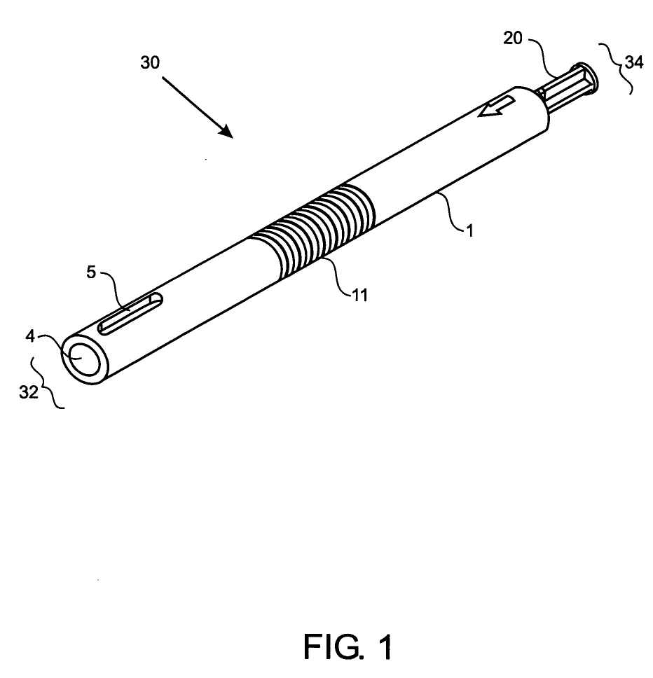

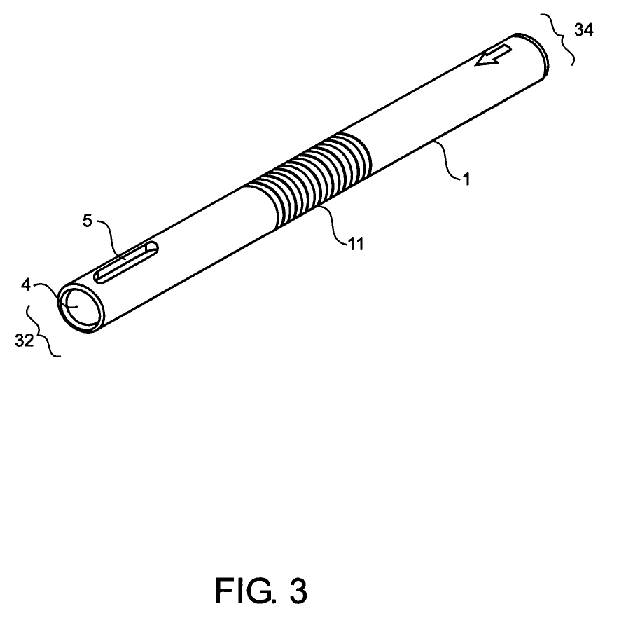

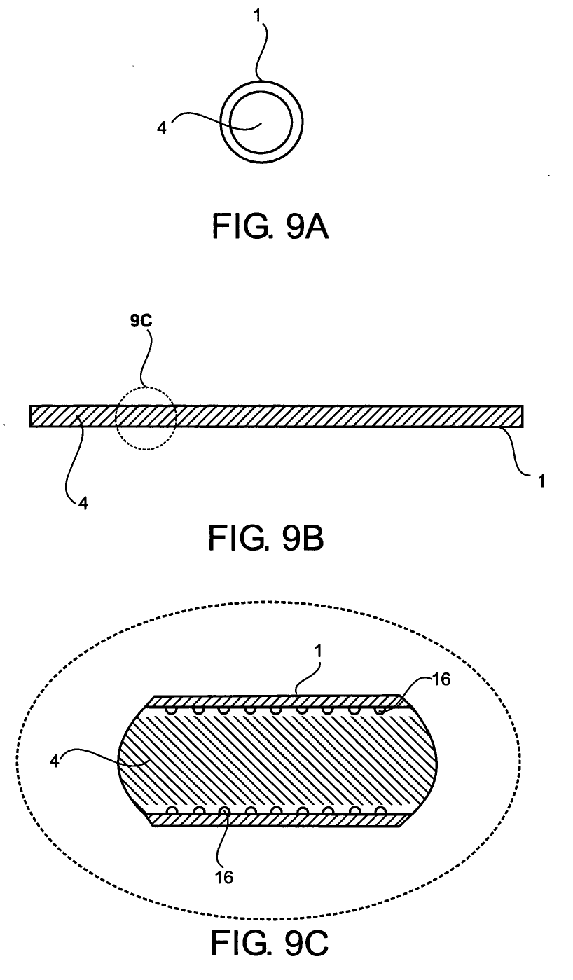

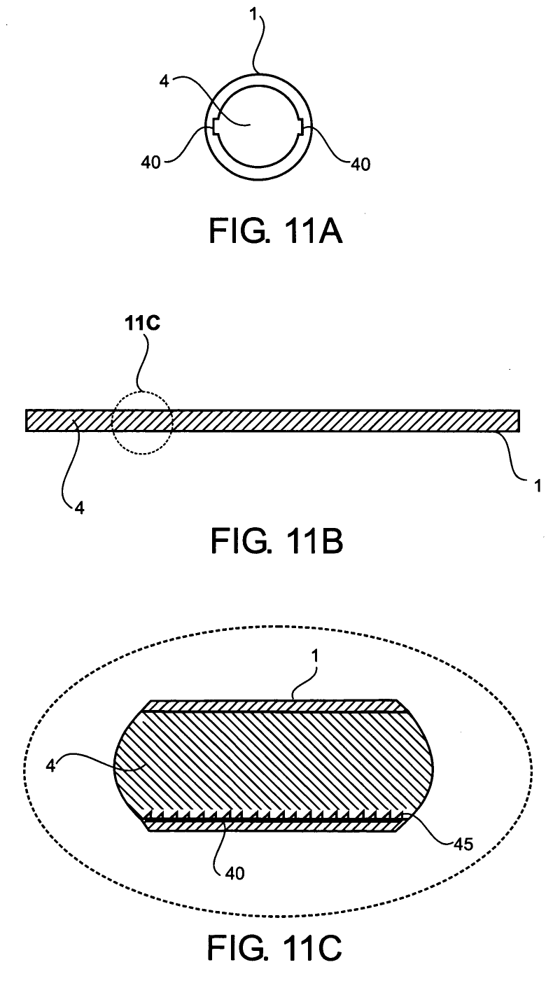

FIG. 1 shows one embodiment of the implant delivery device 30 of the present invention having a proximal end 34 and a distal end 32. In a preferred embodiment, the delivery device 30 has a length suitable for arthroscopic use, i.e., approximately six to about eight inches. The implant delivery device 30 includes a hollow tubular outer shaft 1 (also shown in FIG. 3) having an internal bore 4 along the longitudinal axis. The internal bore 4 extends the entire length of the outer shaft 1 from the distal end 32 to the proximal end 34. FIGS. 9A-9C and FIGS. 11A-11C also illustrate the internal bore 4. The distal end 32 of the outer shaft 1 can have one or more slots 5 through the outer shaft 1 for visualizing the implant (not shown in FIG. 1) when the implant is in the delivery device 30. Slots 5 can be any shape that allows the implant to be visualized while disposed in the delivery device 30 and can be covered with transparent material.

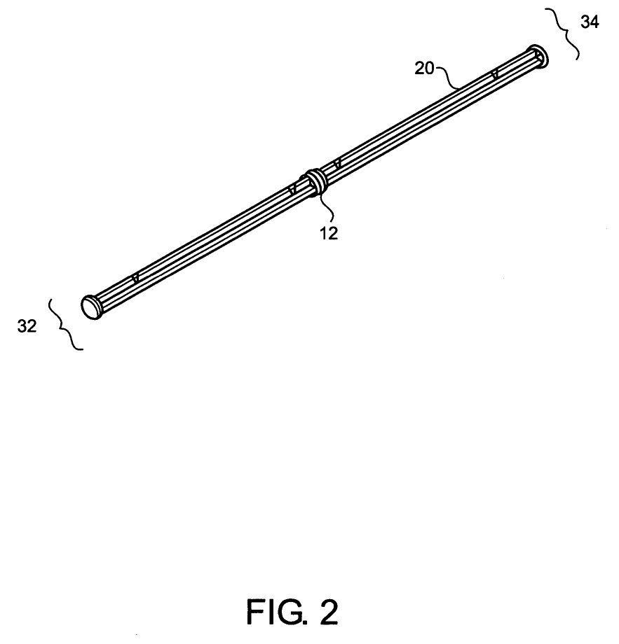

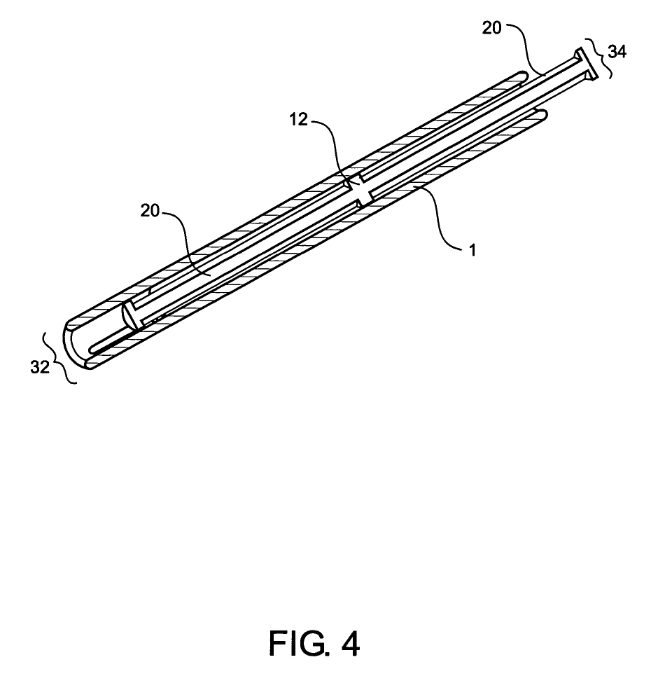

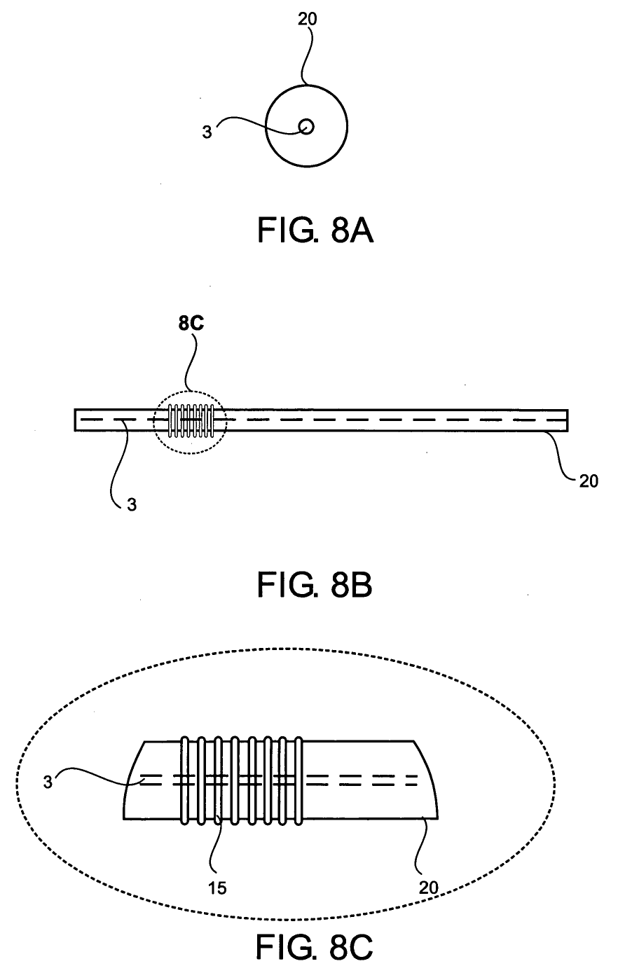

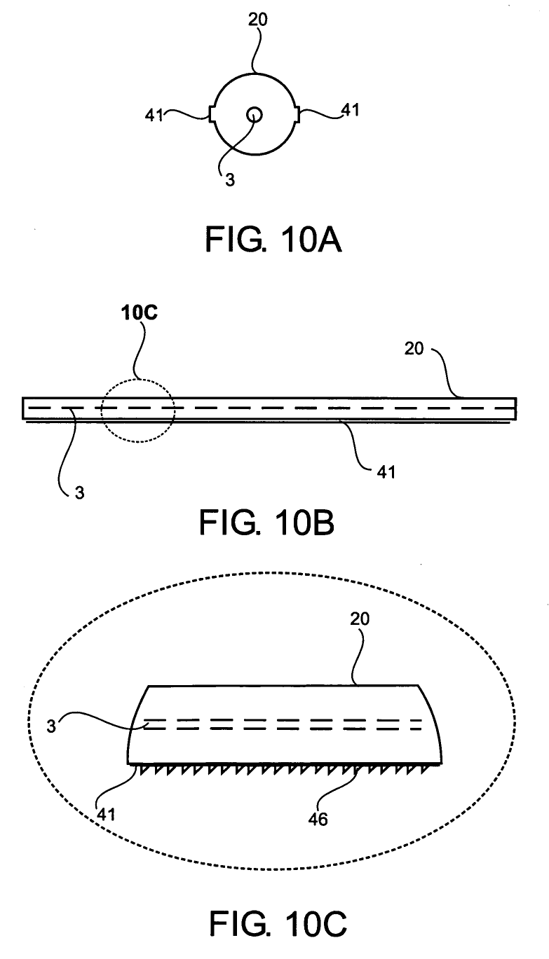

The delivery device 30 illustrated in FIG. 1 further comprises an inner shaft 20 also having proximal and distal ends. The inner shaft 20 is situated within the outer shaft 1 and is able to move proximally and distally through the internal bore 4. FIG. 4 shows a cross-section of delivery device 30 with the inner shaft 20 disposed within the internal bore 4 of the outer shaft 1. As shown in FIGS. 2 and 4, inner shaft 20 has a friction member 12 which contacts the inner surface of the outer shaft 1. Optionally, the inner shaft may contain a small cannula 3 through its center, as shown in FIGS. 8A-8C and 10A-10C. A guide wire attached to the defect by a means such as suturing may be threaded through cannula 3.

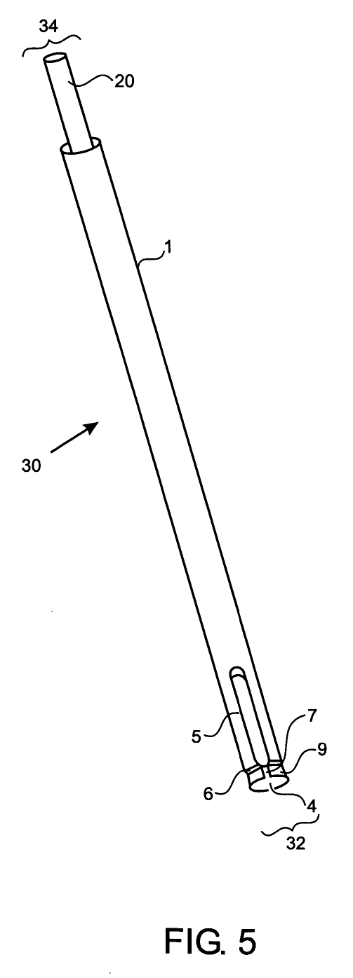

FIG. 5 shows another embodiment of the present invention where the distal end 32 of the delivery device 30 has a small groove 6 running around the outside of the outer shaft 1. In this embodiment, items can attach to the distal end 32 of the outer shaft 1 by having a diameter slightly larger than the outer diameter of the outer shaft 1, fitting over the distal end 32 of the outer shaft 1, and having one or more beads or a rim that snap into the groove 6, thus securing the position of the attached item.

FIG. 5 also shows the delivery device 30 having thin longitudinal slits 7 cut through the distal end 32 of the outer shaft 1 creating leaves 9. Leaves 9 are the sections of the outer shaft 1 between the longitudinal slits 7. The leaves 9 can be made so that they taper slightly inward creating slight compression on the implant (not shown) while in the device 30.

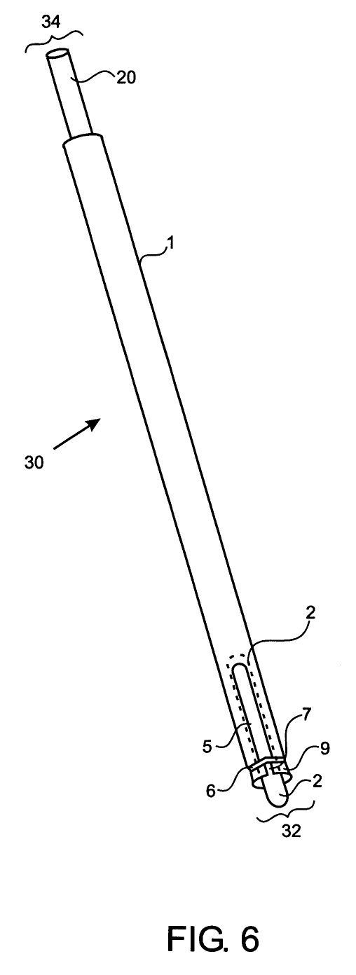

FIG. 6 shows the implant delivery device 30 illustrated in FIG. 5 with an implant 2 disposed in the distal end 32 of the outer shaft 1. In this figure, a portion of the implant 2 extends beyond the distal end 32 of the outer shaft 1 and would have to be cut.

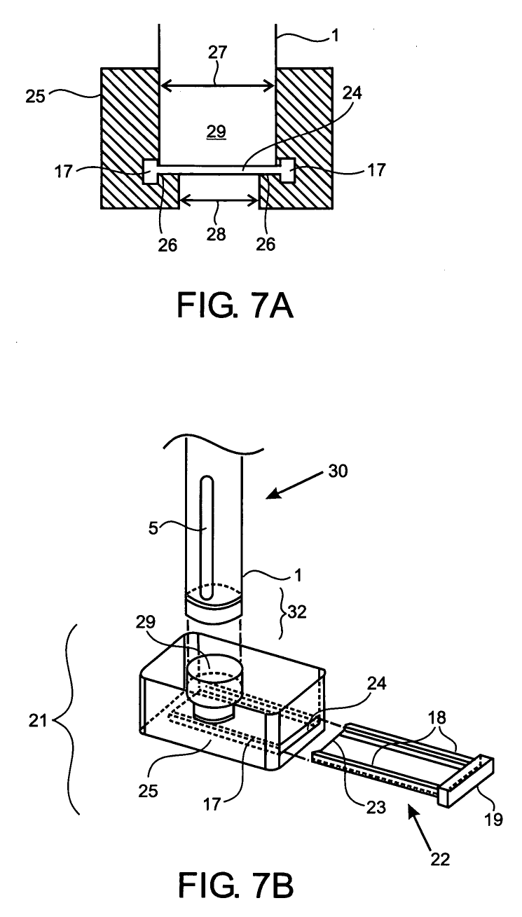

FIGS. 7A and 7B show a preferred embodiment of a cutting device 21 comprising a rectangular base 25 and a cutting blade 22. Rectangular base 25 has a vertical circular hole 29 extending through the base 25 from top to bottom, having an upper diameter 27 and lower diameter 28. The upper diameter 27 is slightly larger than the outer diameter of the outer shaft 1 of the device 30. The lower diameter 28 is slightly less than the outer diameter of the outer shaft 1 but slightly larger than the diameter of implant 2 shown in FIG. 6. Within the hole 29, a shoulder 26 is formed where the upper diameter 27 meets the lower diameter 28. A cutting slot 24 horizontally extends from one side of base 25 and perpendicularly intersects hole 29 at shoulder 26. The sides of cutting slot 24 vertically expand into guide slots 17.

A cutting blade 22 with a sharp cutting edge 23 fits within the cutting slot 24 and can be advanced through cutting slot 24 until the cutting edge 23 is completely advanced across the hole 29. Opposite and parallel to cutting edge 23, cutting blade 22 has a handle edge 19, which has a greater height and width than cutting edge 23. Handle edge 19 is not sharp and is suitable for holding onto by hand. Cutting blade 22 also has two guide edges 18, which intersect and extend from cutting edge 23 to handle edge 19. Guide edges 18 have a greater height than cutting edge 23 and fit into guide slots 17 to provide a secure insertion of cutting blade 22 into cutting slot 24.

To use the implant delivery device 30 in one embodiment of the present invention, the inner shaft 20 is placed within the internal bore 4 of the outer shaft 1 so that no portion of the inner shaft 20 protrudes from the outer shaft 1. An implant 2, which can be a synthetic implant or a transplant of healthy bone or cartilage, is inserted into the distal end 32 of the outer shaft 1. This pushes inner shaft 20 through internal bore 4 toward proximal end 34. As a result, a portion of inner shaft 20 will protrude from proximal end 34 of outer shaft 1. The portion of inner shaft 20 that protrudes from proximal end 34 of outer shaft 1 will be the same length as implant 2 within distal end 32 of outer shaft 1.

The portion of inner shaft 20 that protrudes from the proximal end 34 of the outer shaft is then inserted into a defect. When the proximal end 34 of the inner shaft 20 contacts the bottom of the defect, outer shaft 1 is proximally advanced until the proximal end 34 of the outer shaft 1, which has a larger diameter than inner shaft 20 and the defect, is level with and contacts the surface of the tissue surrounding the defect. This act displaces inner shaft 20 through internal bore 4 toward distal end 32 of outer shaft 1, causing a portion of the implant 2 to extend beyond the distal end 32 of outer shaft 1.

The protruding end of implant 2, i.e., the portion of implant 2 extending beyond the distal end 32 of the outer shaft 1, is then cut off. In one embodiment, a knife is used to cut implant 2. In another embodiment, the cutting device 21 illustrated in FIGS. 7A and 7B is used. To use cutting device 21, the distal end 32 of outer shaft 1 is inserted through vertical hole 29 in base 25 until outer shaft 1 contacts shoulder 26. The shoulder 26 prevents outer shaft 1 from advancing further through hole 29, but because the lower diameter 28 is equal to or slightly larger than the diameter of internal bore 4, the portion of implant 2 that extends beyond the distal end 32 of the outer shaft 1 passes through vertical hole 29 beyond the shoulder 26. Cutting blade 22 is inserted into cutting slot 24 and advanced until cutting edge 23 horizontally intersects vertical hole 29 and cuts through implant 2. The cutting device 21 is removed after cutting off the protruding portion of the implant.

The device 30 can be removed from the defect prior to or immediately after cutting off the excess implant material. Once removed from the defect, implant delivery device 30 is flipped around so that the distal end 32 of the device 30 is oriented toward the defect. The distal end 32 of outer shaft 1 is placed over the defect. The inner shaft 20 is advanced through the internal bore 4 towards distal end 32, pushing the remaining portion of implant 2 into the defect. The defect, if intentionally created, is formed with a diameter such that implant 2 completely fills the defect.

Another embodiment (not shown) of cutting device 21 comprises hole 29 having a diameter slightly less than the outer diameter of outer shaft 1 but slightly larger than the diameter of implant 2. In this embodiment, the portion of implant 2 that extends beyond the distal end 32 of outer shaft 1 can be inserted into hole 29 but the distal end 32 of outer shaft 1 cannot be inserted into hole 29. Guide slots 17 are disposed into the top surface of base 25. Guide edges 18 of cutting blade 22 fit into guide slots 17 allowing cutting blade 22 to slide along the top surface of base 25 until cutting edge 23 cuts through implant 2 at the top of hole 29.

Another embodiment (not shown) of cutting device 21 comprises hole 29 having a diameter slightly larger than the outer diameter of outer shaft 1 until hole 29 reaches the bottom surface of base 25. At the bottom surface of base 25, hole 29 has a diameter slightly less than the outer diameter of outer shaft 1 but slightly larger than the diameter of implant 2. In this embodiment, the portion of implant 2 that extends beyond the distal end 32 of outer shaft 1 can exit through the bottom of hole 29 but the distal end 32 of outer shaft 1 cannot. Guide slots 17 are disposed into the bottom surface of base 25. Guide edges 18 of cutting blade 22 fit into guide slots 17 allowing cutting blade 22 to slide along the bottom surface of base 25 until cutting edge 23 cuts through implant 2 at the bottom of hole 29.

FIGS. 8A-8C show an embodiment of this invention wherein a section of inner shaft 20 comprises ridges 15. Ridges 15 are raised rings around a portion of the outer surface of inner shaft 20. In this embodiment, friction beads 16 are also disposed on the corresponding section of the inner surface of outer shaft 1, as shown in FIGS. 9A-9C. The friction beads 16 are raised higher than the surrounding inner surface of outer shaft 1. During proximal and distal movement of inner shaft 20 through internal bore 4 of outer shaft 1, friction beads 16 engage with ridges 15 requiring extra force to continue to advance the inner shaft 20 through the internal bore 4. By “engage with” it is meant that friction beads 16 or serrated teeth 45, as described below, on the inner surface of the outer shaft 1 come into physical contact with ridges 15 or serrated teeth 46, as described below, on the inner shaft 20 providing extra resistance against movement of inner shaft 20 through the internal bore 4.

FIGS. 10A-10C show another embodiment of this invention wherein the outer surface of inner shaft 20 contains at least one alignment rib 41 along the length of inner shaft 20. As shown in FIG. 10A, an alignment rib 41 is a section of the outer surface of inner shaft 20 raised higher than the surrounding surface. Serrated teeth 46 extend out from a section of the alignment rib 41.

Also in this embodiment, as shown in FIGS. 11A-11C, the outer shaft 1 has at least one alignment slot 40 cut into its inner surface. The depth, position, and number of alignment slots 40 correspond to the height, position, and number of alignment ribs 41 on inner shaft 20 so that the alignment ribs 41 of inner shaft 20 fit into the alignment slots 40 of the inner surface of outer shaft 1. Serrated teeth 45 extend out from a section of alignment slots 40. The section of alignment slot 40 that contains the serrated teeth 45 corresponds to the section of the alignment rib 41 that contains serrated teeth 46.

In this embodiment, inner shaft 20 fits in the internal bore 4 of the outer shaft 1 when alignment rib 41 is aligned with alignment slot 40. During proximal and distal movement of inner shaft 20 through internal bore 4 of outer shaft 1, the serrated teeth 46 along alignment rib 41 contact and engage with serrated teeth 45 along alignment slot 40 preventing unwanted movement.

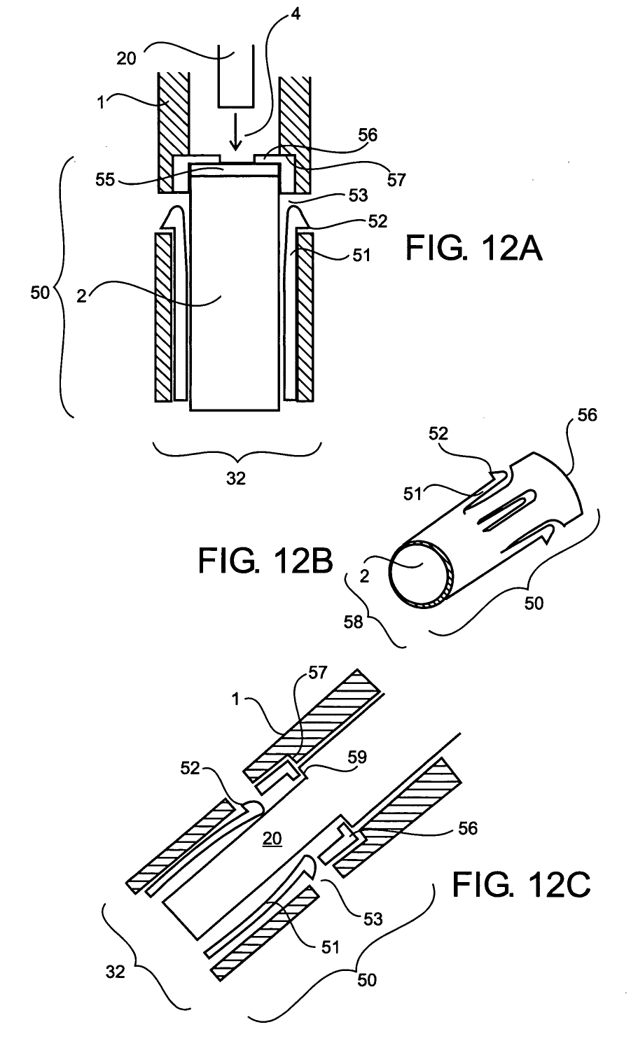

FIGS. 12A-12C illustrate a capsule loader 50 that can be used with implant delivery device 30. The capsule loader 50 is a hollow tube having an outer diameter slightly less than the inner diameter of outer shaft 1 allowing the capsule loader 50 to fit within internal bore 4 at the distal end 32 of outer shaft 1. Optionally, the inner diameter of outer shaft 1 may be decreased along internal bore 4 creating internal shoulder 57. The outer diameter of the capsule loader 50 is great enough that when inserted into outer shaft 1, the capsule loader 50 contacts internal shoulder 57 and is prevented from proximally advancing further through internal bore 4. Preferably internal shoulder 57 is positioned proximally from the distal end 32 of the outer shaft 1 at a distance equal to the length of capsule loader 50 so that when capsule loader 50 contacts internal shoulder 57 the front end 58 of capsule loader 50 is flush with the distal end 32 of the outer shaft 1.

The capsule loader 50 has an inner diameter slightly greater than the diameter of inner shaft 20. The inner diameter of capsule loader 50 is also slightly greater than implant 2, allowing implant 2 to be disposed within capsule loader 50. The back end 56 of capsule loader 50 has a round hole (also called an “opening”) therethrough with a diameter slightly less than the rest of the capsule loader 50 but slightly greater than the diameter of distal end 32 of the inner shaft 20, thus allowing inner shaft 20 to pass through capsule loader 50. Optionally, the diameter of inner shaft 20 is increased at a point proximal from the distal end 32 of the inner shaft 20, preferably at a distance from the distal end 32 of the inner shaft 20 equal to the length of the capsule loader 50, to form shoulder 59. The increased diameter of the inner shaft 20 at shoulder 59 remains less than the inner diameter of the outer shaft 1 but is greater than the diameter of the back end 56 of capsule loader 50. When distally advanced within outer shaft 1, the inner shaft 20 passes through capsule loader 50 until shoulder 59 contacts the back end 56 of capsule loader 50 as shown in FIG. 12C.

The capsule loader 50 contains a backplate 55, which has a diameter slightly less than the inner diameter of the capsule loader 50 allowing it to proximally and distally move through the capsule loader 50. The backplate 55 has a greater diameter than the back end 56 of capsule loader 50. When an implant 2 is disposed within capsule loader 50, the backplate 55 is between implant 2 and the back end 56 of capsule loader 50.

The capsule loader 50 also has at least one flexible leaflet 51. Flexible leaflets 51 are projections on the outer surface of capsule loader 50 that run along the longitudinal axis thereof. Flexible leaflets 51 can be pressed inward but return to their original position when the inward pressure is released. On the ends of the flexible leaflets are prongs 52, which extend outward from capsule loader 50. When the flexible leaflets are not pressed inward, capsule loader 50 cannot be inserted into the outer shaft 1 because prongs 52 do not fit within internal bore 4. When the flexible leaflets 51 are pressed inward, the prongs 52 fit within internal bore 4 of outer shaft 1 and the capsule loader 50 can be inserted.

In conjunction with use of capsule loader 50, there is at least one prong hole 53 cut through outer shaft 1. The dimensions of the prong holes 53 are slightly larger than prongs 52 such that the prongs 52 can fit through prong holes 53. Preferably prong holes 53 are at a distance from the distal end 32 of the outer shaft 1 so that the prongs 52 are aligned with the prong holes 53 when the capsule loader 50 is inserted into outer shaft 1 and the front end 58 is flush with distal end 32 of outer shaft 1.

To use the capsule loader 50 with the implant delivery device 30, the back end 56 of capsule loader 50 with implant 2 already disposed therein is inserted into the distal end 32 of outer shaft 1. To allow the capsule loader 50 to be inserted into internal bore 4, flexible leaflets 51 must be pressed inward. Once the capsule loader 50 is inserted into outer shaft 1 and the inward pressure is released, the flexible leaflets 51 will exert an outward pressure against the inner surface of outer shaft 1. When prongs 52 on the end of flexible leaflets 51 are aligned with prong holes 53 in outer shaft 1, the outer pressure exerted by flexible leaflets 51 will move the prongs 52 into prong holes 53. While prongs 52 are in the prong holes 53, unwanted motion of the capsule loader 50 is prevented. In addition, the capsule loader 50 may be prevented from further proximal movement through internal bore 4 by internal shoulder 57.

Because the diameter of the distal end 32 of inner shaft 20 is slightly less than the diameter of the hole in back end 56 of capsule loader 50, the distal end 32 of inner shaft 20 can be distally advanced through back end 56 and then through capsule loader 50. While distally advancing through capsule loader 50, inner shaft 20 contacts backplate 55 and pushes backplate 55 and implant 2 distally through capsule loader 50. Continued distal movement by inner shaft 20 will push implant 2 out through front end 58 of capsule loader 50 and out through distal end 32 of outer shaft 1 of delivery device 30. When shoulder 59 of inner shaft 20 contacts back end 56 of capsule loader 50, inner shaft 20 cannot be distally advanced further through capsule loader 50.

After implant 2 has been expelled, capsule loader 50 is removed from delivery device 30 by pushing inward on prongs 52 through prong holes 53 while simultaneously pushing inner shaft 20 toward distal end 32. The prongs 52 are pushed out of prong holes 53 and the shoulder 59 of inner shaft 20 will push against the back end 56 of capsule loader 50. Because the prongs 52 no longer hold capsule loader 50 in place, the capsule loader 50 will be pushed out through the distal end 32 of outer shaft 1.

While the invention has been described with certain preferred embodiments, it is understood that the preceding description is not intended to limit the scope of the invention. It will be appreciated by one skilled in the art that various equivalents and modifications can be made to the invention shown in the specific embodiments without departing from the spirit and scope of the invention.

1. A bone or cartilage implant delivery device comprising:
a tubular outer shaft having a proximal and distal end, a longitudinal axis, and an internal bore along the longitudinal axis of said outer shaft;
an inner shaft having a distal end and a proximal end suitable for insertion into a defect, said inner shaft adapted to fit within said internal bore of the outer shaft so that the inner shaft and the outer shaft are slidably engaged.

2. The device of claim 1 wherein one or more of the shafts comprise means to provide friction-retarded movement of the inner shaft through the outer shaft.

3. The device of claim 1 also comprising an implant disposed within the distal end of said outer shaft.

4. The device of claim 1 wherein the inner shaft has a cannula through its center.

5. The device of claim 3 also comprising at least one slot in the distal end of the outer shaft for visualizing said implant.

6. The device of claim 1 wherein the distal end of the outer shaft comprises tapered leaves.

7. The device of claim 1 further comprising a snap-bead groove disposed on the distal end of the outer shaft.

8. The device of claim 1 further comprising smooth, rounded surfaces on the proximal and distal ends of the outer shaft and inner shaft.

9. The device of claim 1 wherein a section of the inner surface of the outer shaft has serrated teeth frictionally enageable with serrated teeth on a corresponding section of the outer surface of the inner shaft.

10. The device of claim 1 wherein a section of the inner surface of the outer shaft has beads disposed thereon and a corresponding section of the outer surface of the inner shaft has ridges disposed thereon so that when the inner shaft is moved distally or proximally through the outer shaft, the beads frictionally engage with the ridges.

11. An implant cutting device for cutting off a protruding end of an implant disposed within the implant delivery device of claim 1, said cutting device comprising a base comprising a vertical hole therethrough for receiving said protruding end of said implant.

12. The implant cutting device of claim 11 wherein said vertical hole adapted for receiving said protruding end of said implant has an upper diameter slightly larger than the outer diameter of said shaft and a lower diameter less than the outer diameter of said shaft.

13. The implant cutting device of claim 11 also comprising means for receiving at least one cutting blade.

14. The implant cutting device of claim 11 also comprising at least one cutting blade adapted to slide within said means for receiving at least one cutting blade and cut off the protruding end of said implant.

15. The implant cutting device of claim 14 wherein said at least one cutting blade intersects said vertical hole at the point where said upper diameter meets said lower diameter.

16. An implant capsule loader for inserting an implant into the outer shaft of the implant delivery device of claim 1, said capsule loader comprising:
a hollow tube having a front end and a back end with an opening therethrough, and adapted to fit within the distal end of said outer shaft.

17. The capsule loader of claim 16 comprising a backplate disposed within said hollow tube covering the opening in the back end of said tube.

18. The capsule loader of claim 16 also comprising at least one flexible leaflet along the outer surface of said hollow tube fixed at the front end of said hollow tube and having a free end toward the back end of said hollow tube, said flexible leaflet having an outwardly-extending prong at the free end thereof; said prong being adapted to fit within a hole in said outer shaft.

19. The capsule loader of claim 18 comprising a plurality of flexible leaflets.

20. The capsule loader of claim 16 comprising an implant disposed therein.

21. An implant delivery system comprising:
an implant delivery device comprising a hollow outer shaft, and an inner shaft movably disposed therein; and an implant cutting device comprising means for receiving an implant protruding from said outer shaft and a cutting blade for cutting off said protruding portion.

22. The implant delivery system of claim 21 also comprising an implant capsule loader comprising a hollow tube adapted to contain an implant and to fit within and be attached to an end of said hollow shaft.

23. The implant delivery system of claim 22 also comprising an implant.

24. A method for delivering a bone or cartilage implant into a defect in a tissue having an unmeasured depth using the implant delivery device of claim 1 comprising the steps:
inserting said implant into the distal end of said loading device, wherein when said implant is disposed in said loading device the proximal end of the inner shaft protrudes from the proximal end of the outer shaft and the length of said implant and equals the length of the protruding section of the inner shaft;
inserting the proximal end of the inner shaft into the defect until the proximal end of the inner shaft contacts the bottom of the defect;
advancing the outer shaft in the proximal direction until the proximal end of the outer shaft contacts the surface of tissue surrounding the defect, causing a portion of the implant to extend beyond the distal end of the outer shaft;
cutting off the portion of the implant extending beyond the distal end of the outer shaft, leaving a remaining portion disposed within the outer shaft;
placing the distal end of the loading device over the defect; and
distally advancing the inner shaft to push the portion of the implant remaining after cutting into the defect.

25. The method of claim 24 further comprising placing a cap around the distal end of the outer shaft after the portion of the implant extending beyond the distal end of the outer shaft has been cut off and adding a bioactive fluid to the distal end of said outer shaft.

26. A kit comprising at least one bone or cartilage implant delivery device, said implant delivery device comprising:
a tubular outer shaft having a proximal and distal end, a longitudinal axis, and an internal bore along the longitudinal axis of said outer shaft; and
an inner shaft having a distal end and a proximal end suitable for insertion into a defect, said inner shaft adapted to fit within said internal bore of the outer shaft so that the inner shaft and the outer shaft are slidably engaged.

27. The kit of claim 26 further comprising an implant.

28. The kit of claim 26 further comprising a knife.

29. The kit of claim 26 comprising a plurality of bone or cartilage implant delivery devices each having different sizes of internal bores and inner shafts.
